(19) United States
(12) Patent Application Publication
(10) Pub. No.: US 2009/0257971 A1
(43) Pub. Date: Oct. 15, 2009

(54) Antiperspirant composition

(76) Inventor: Keith Roberts, Stillwater, MN (US)

Correspondence Address: JACOBSON & JOHNSON, ONE WEST WATER STREET, SUITE 285, ST. PAUL, MN, 55107, US

(21) Appl. No.: 10/536,116

(22) Filed: Mar. 9, 2007

Related U.S. Application Data
(63) Continuation of application No. 10/422,438, filed on Apr. 24, 2003, now abandoned.

(51) Int. Cl. A61K 8/02; A61P 17/00

(52) U.S. Cl. 424/68; 424/65

(57) ABSTRACT

An antiperspirant composition with the antiperspirant composting containing a thickening agent in sufficient quantity so as to produce an antiperspirant composition wherein the viscosity is less than 2400 centpoises.

### FIELD OF THE INVENTION

This invention relates generally to antiperspirants and more particularly to antiperspirant compositions for treating an excessive sweating condition known as hyperhidrosis.

### CROSS REFERENCE TO RELATED APPLICATIONS

None

### STATEMENT REGARDING FEDERALLY SPONSORED RESEARCH OR DEVELOPMENT

None

### REFERENCE TO A MICROFICHE APPENDIX

None

### BACKGROUND OF THE INVENTION

The problem of excessive localized body sweating is known as hyperhidrosis. The condition causes portions of the body to sweat excessively, particularly in areas such as face, underarms, hands and feet. Generally, over-the-counter antiperspirants are insufficient to stop the excess sweating. As a result, different methods and compositions have been developed to address the problem of excess body sweating.

A surgical procedure called endoscopic transthoracic sympatheectomy is used to treat excess sweating on the palms of a persons hand. While this procedure can reduce sweating on a persons hands it can cause increase sweating on other parts of the body.

Another surgical procedure involves Botox injections which can stop sweating for up to six to eight months. However, the Botox injection may have unwanted side effects.

Another treatment is an electrical device known as the Drionic machine that plugs up the sweat pores. After several applications it can reduce sweating for up to four to six weeks. It can cause tingling sensation that patients find unpleasant.

Another method of treating excessive sweating involves a person ingesting an oral medication such as Robinul™.

Daily U.S. Pat. No. 2,814,584 discloses a buffered antiperspirant composition that contains aluminum chloride in the amount of about 20% which he states is generally accepted as being an effective antiperspirant. Urea is used as a buffering agent with the composition having an aqueous base.

Daily U.S. Pat. No. 2,814,585 dices an antiperspirant composition which he is non-irritating to the skin and non corrosive to textiles. As the active ingredient Daley uses Zirconyl chloride and aluminum chlohydroxide complex in an aqueous base.

Grad U.S. Pat. No. 2,854,382 discloses an antiperspirant composition. Grad points out that buffering agents drastically reduce the effect of strongly acid salts of aluminum and that the compositions can be buffered without decreasing the antiperspirant efficacy. Grad proposes the use of aluminum chlorhydroxide that can range from about 5% to about 30% which are in combination with zirconyl hydroxy chloride.

Shin et al U.S. Pat. No. 4,774,079 discloses a further antiperspirant composition that uses a combination of aluminum chlohydrate, aluminum chloride and an aluminum zirconium polychlohydrate complex which includes a buffering agent such as glycine.

One commercial available product which can be applied with a roll-on applicator uses aluminum chloride as the active ingredient in an aqueous base and is known as Certain Dri®. The product is applied to dry skin at bed time and is washed off in the morning. The treatment is repeated until the excess sweating is under control. One of the difficulties with the product is that the composition can drip out of the applicator bottle.

Another product commercially available antiperspirant composition utilizing aluminum chloride is Maxim™ which is sold by Corad Healthcare, Inc. The Maxim antiperspirant composition also includes aluminum chloride as the active agent in an aqueous base and a buffering agent to control the pH of the composition. In addition, Maxim utilizes a thickening agent to prevent the composition from dripping from the bottle as well as to maintain the aluminum chloride at the site of the excess sweating.

The purpose of the thickening agent is to improve the adhesion and the effectiveness of the antiperspirant composition as well as makes it less likely that the antiperspirant composition will spill or leak out of the container.

The present invention comprises a discover that by limiting the amount of thickening agent used with the antiperspirant to lower the viscosity of the antiperspirant composition one can at the same time increase the effectiveness of the active ingredient aluminum chloride. While it is not fully understood it is believed a two fold effect occurs, first it appears pore blockage may be caused by the thickening agent in the composition can be minimized and second, the effectiveness of the aluminum chloride is enhanced. In addition, it has been found by maintaining the viscosity of the antiperspirant composition within a limited range one simultaneously decreases the drying time of the antiperspirant from hours to minutes and also prevent the antiperspirant composition from feeling greasy or oily on the persons body. At the same time it has been found that an antiperspirant composition of lesser viscosity can also prevent unwanted liquid spills or leaks from the dispenser.

### SUMMARY OF THE INVENTION

An antiperspirant composition with the antiperspirant composting containing a thickening agent in sufficient quantity so as to produce an antiperspirant composition wherein the viscosity of the antiperspirant composition is less than 2400 centpoises.

### BRIEF DESCRIPTION OF THE DRAWINGS

FIG. 1 shows a roll-on applicator contain the antiperspirant composition of the present invention.

### DESCRIPTION OF THE PREFERRED EMBODIMENT

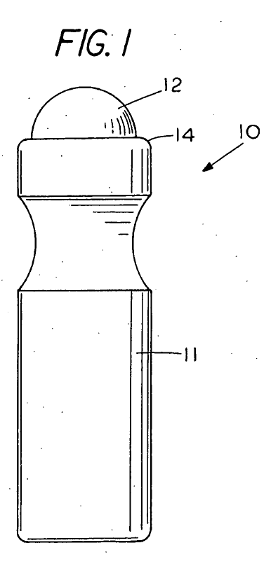

FIG. 1 shows a front view of a roll-on applicator 10 having a first end with a base 11 for a user to grasp and a second end a ball 12 which is captured by an annular lip 14 to maintain the ball 12 in a rollable relationship with the container. The antiperspirant applicator contains an antiperspirant composition that a user applies to portions of the body that sweat excessively due to the condition known as hyperhidrosis. The antiperspirant composition, which is picked up on the surface of the ball 12 as it rotates through base 11 includes aluminum chloride as the active ingredient in an aqueous base. In order to maintain the pH at levels not to cause skin irritation a buffering agent such as amino methyl propanol is added to the antiperspirant composition.

One of the difficulties with the use of roll-on applicators is that the in order to prevent dripping and maintain the aluminum chloride at the site being treated a thickening agent has been added to the composition. While it has been found that the thickening agent prevents the antiperspirant composition from accidentally spilling out of the applicator it has been discovered that the thickening agent in the antiperspirant composition can actually reduce the effectiveness of the aluminum chloride. While the mechanism is not completely understood it is believed that the thickening agent may block some of the pores thereby reducing the effectiveness of the antiperspirant composition.

In the present invention it has been discovered that by limiting the amount of thickening agent in the antiperspirant so as to maintain that viscosity of the antiperspirant composition at less than 2400 centipoises and preferably in the range of 600 to 1000 centipoises and ideally about 800 centipoises provides an improved antiperspirant composition. The reduction of viscosity of the antiperspirant composition provides a user benefit in that the user can now tell if there is antiperspirant remaining in the applicator since shaking the applicator dispenser produces a sloshing sound if there is antiperspirant in the applicator dispenser. In addition, it has been found that thickening agent at lesser amounts can prevent the antiperspirant composition from leaking out between the roll-on ball and the seat for the roll-on ball. In addition, to the consumer benefits it has been found that the reduction of thickening agent to less than 2400 centipoises increases the effectiveness of the aluminum chloride thereby enabling use of lesser concentrations of aluminum chloride and thus potentially minimizing skin irritation caused by higher concentrations of aluminum chloride.

Thus the present invention comprises a roll-on antiperspirant applicator including: an antiperspirant composition located in the roll-on applicator, an aqueous base in the antiperspirant composition, an active ingredient in the aqueous base, the active ingredient comprising aluminum chloride, a buffering agent in the antiperspirant composition, with the improvement comprising a thickening agent in the composition in quantities sufficient to maintain the antiperspirant composition with a viscosity of less than 2400 centipoises and preferably in the range of 600-1000 centipoises.

In the present invention an antiperspirant composition was prepared by mixing 6% by weight of aluminum chloride in an aqueous base. A buffering agent of amino methyl propanol was added in sufficient quantity to increase the pH to a level of 2.5. A thickening agent, Polyquaterenium-37 was added so as to increase the viscosity of the composition sufficient to inhibit spillage or leakage when used in a roll-on applicator but in insufficient quantity to raise the viscosity of the antiperspirant composition above 800 centipoises. Thickening agent are known in the art and a list of such thickening agents is disclosed in U.S. Pat. No. 4,198,316 and U.S. Pat. No. 5,958,397. In the present antiperspirant composition the preferred thickening agent is Polyquaterenium-37, a quaternized polyacrylic thickener sold under the trade name Syntalen CR from the 3V Corporation is used.

The antiperspirant composition which was placed in an applicator dispenser has been found that the rollable ball which is rotatable carried by the container will not leak antiperspirant composition therefrom if the viscosity of the antiperspirant composition is in the range of 600 to 1000 centipoises and at the same time the antiperspirant composition dries in a matter of minutes.

Although the antiperspirant composition is described in relation to a roll-on ball type applicator other methods of applying the antiperspirant composition can also be used.

1-11. (canceled)

12. A method of treating an excessive sweating condition known as hyperhidrosis comprising the steps of:
mixing an antiperspirant solution containing a range of about 5% to about 15% by weight of aluminum chloride in an aqueous base;
adding a buffering agent comprising amino methyl propanol to the solution to increase the pH of the solution to a level of at least 2.5;
adding a thickening agent comprising Polyquaterenium-37 to the solution to maintain an in use viscosity of the antiperspirant composition of less than 800 centipoises;
filling a container housing with the antiperspirant solution;
attaching a rotatable ball to the container housing to form a roll-on applicator wherein the clearance between the ball and the container housing carrying the rotatable ball is sufficiently small so that the antiperspirant composition having viscosity of 600 centipoises will not leak therepast; and
using the roll-on applicator to apply the antiperspirant solution to portions of the body that sweat excessively due to the condition known as hyperhidrosis.

13. A method of treating an excessive sweating condition known as hyperhidrosis comprising the steps of:
mixing an antiperspirant solution containing 6% by weight of aluminum chloride in an aqueous base;
adding a buffering agent comprising amino methyl propanol to the solution to increase the pH of the solution to a level of 2.5;
adding a thickening agent comprising Polyquaterenium-37 to the solution to maintain an in use viscosity of the antiperspirant composition of at least 600 centipoises;
filling a container with the antiperspirant solution;
attaching a rotatable ball to the container to form a roll-on antiperspirant applicator wherein the clearance between the ball and the container carrying the rotatable ball is sufficiently small so that the antiperspirant composition having a viscosity of 600 centipoises will not leak therepast; and
using the roll-on applicator to apply the antiperspirant solution to portions of the body that sweat excessively due to the condition known as hyperhidrosis.

14. A roll-on antiperspirant applicator including:
a container;
a roll-on ball applicator rollable mounted in said container;
an antiperspirant composition located in said container;
an aqueous base in said antiperspirant composition;
an active ingredient in the aqueous base, said active ingredient comprising aluminum chloride in a range of about 5% to 15% by weight;
a buffering agent in said antiperspirant composition, said buffer agent comprising amino methyl propanol in sufficient quantity to increase the pH of the antiperspirant composition to at least 2.5;
with the improvement comprising a thickening agent comprising Polyquaterenium-37 in said composition in quantities sufficient to maintain an in use viscosity of the antiperspirant composition less than 800 centipoises to increase the effectiveness of the aluminum chloride and wherein the clearance between the rotatable ball and the container housing carrying the rotatable ball is sufficient small to prevent the less than 800 centipoises antiperspirant composition from leaking therepast.
